(19) United States
(12) Patent Application Publication
(10) Pub. No.: US 2017/0049340 A1
(43) Pub. Date: Feb. 23, 2017

(54) METHOD AND APPARATUS FOR MEASURING PULSE WAVE DELIVERY VELOCITY DIFFERENCE BY MOBILE DEVICE

(71) Applicant: SAMSUNG ELECTRONICS CO., LTD., Gyeonggi-do (KR)

(72) Inventors: Shin-hee CHO, Suwon-si (KR); Kwang-choon KIM, Suwon-si (KR); Jae-geol CHO, Yongin-si (KR)

(21) Appl. No.: 15/119,066

(22) PCT Filed: Mar. 11, 2014
(86) PCT No.: PCT/KR2014/002022

§ 371 Date: Aug. 15, 2016

(30) Foreign Application Priority Data
Feb. 28, 2014 (KR) 10-2014-0024659

(51) Int. Cl. A61B 5/0285; A61B 5/00; A61B 5/0404; A61B 5/0408; A61B 5/024; A61B 5/026

(52) U.S. Cl. CPC A61B 5/0285; A61B 5/02427; A61B 5/02438; A61B 5/0261; A61B 5/0404; A61B 5/04085; A61B 5/6898; A61B 2560/0468; A61B 2562/0209; A61B 2562/0238; A61B 2562/063

(57) ABSTRACT

A method of measuring a pulse wave velocity difference in a mobile device includes: obtaining electrocardiogram (ECG) information of an object through a first electrode that contacts a first surface of the object and is included in the mobile device and a second electrode that contacts a second surface of the object and is included in the mobile device; obtaining blood flow change information of the first surface and the second surface by using a first optical sensor that contacts the first surface and is included in the mobile device and a second optical sensor that contacts the second surface and is included in the mobile device; obtaining pulse wave velocity information to the first surface and pulse wave velocity information to the second surface based on the obtained ECG information and the obtained blood flow change information of the first surface and the second surface; and measuring pulse wave velocity difference information based on the pulse wave velocity information to the first surface and the second surface.

### TECHNICAL FIELD

The present invention relates to a method and apparatus for measuring a pulse wave velocity difference, and more particularly, to a method and apparatus for measuring a pulse wave velocity in a mobile device.

### BACKGROUND ART

In general, a user in a hospital may predict whether an object has a vascular disease by measuring a blood pressure of the object. When the blood pressure is high or there is a large blood pressure difference between hands, it may be determined that a probability that the object is to get a vascular disease or the object suffers from a vascular disease is high. Since a pulse wave velocity and a blood pressure have similar characteristics, whether the object has a vascular disease may be predicted by measuring a pulse wave velocity.

However, since equipment for measuring a pulse wave velocity is expensive and large and it is difficult to measure a pulse wave velocity by using the equipment, it is difficult to measure a pulse wave velocity at all times.

### DETAILED DESCRIPTION OF THE INVENTION

### Technical Problem

In an embodiment of the present invention, there is provided a method and apparatus for measuring a pulse wave velocity difference.

### Technical Solution

According to an embodiment of the present invention, there is provided a method of measuring a pulse wave velocity difference by using a mobile device.

### Advantageous Effects of the Invention

In an embodiment of the present invention, there is provided a method of measuring a pulse wave velocity difference of an object in a mobile device.

### DESCRIPTION OF THE DRAWINGS

FIG. 1 is a view for explaining an arrangement of electrodes and optical sensors of a mobile device according to an embodiment of the present invention.

FIG. 2 is a view for explaining an arrangement of electrodes and optical sensors of the mobile device according to an embodiment of the present invention.

FIG. 3 is a view for explaining an arrangement of electrodes and optical sensors of the mobile device according to an embodiment of the present invention.

FIG. 4 is a view for explaining a posture for measuring pulse wave velocity difference information by using the mobile device according to an embodiment of the present invention.

FIG. 5 is a flowchart for explaining a method of measuring a pulse wave velocity by using a mobile device according to an embodiment of the present invention.

FIG. 6 is a block diagram for explaining the mobile device according to an embodiment of the present invention.

FIG. 7 is a graph for explaining electrocardiogram (ECG) information according to an embodiment of the present invention.

FIG. 8 is a graph for explaining pulse wave information according to an embodiment of the present invention.

FIG. 9 is a detailed flowchart for explaining a method of measuring pulse wave velocity difference information according to an embodiment of the present invention.

FIG. 10 is a flowchart for explaining a method of outputting a notification message according to pulse wave velocity difference information according to an embodiment of the present invention.

FIG. 11 is a detailed block diagram for explaining the mobile device according to an embodiment of the present invention.

FIG. 12 is a block diagram for explaining an operation of a circuit unit of the mobile device according to an embodiment of the present invention.

### BEST MODE

In an embodiment of the present invention for solving the conventional problems, a method of measuring a pulse wave velocity difference in a mobile device may include: obtaining electrocardiogram (ECG) information of an object through a first electrode that contacts a first surface of the object and is included in the mobile device and a second electrode that contacts a second surface of the object and is included in the mobile device; obtaining blood flow change information of the first surface and the second surface by using a first optical sensor that contacts the first surface and is included in the mobile device and a second optical sensor that contacts the second surface and is included in the mobile device; obtaining pulse wave velocity information to the first surface and pulse wave velocity information to the second surface based on the obtained ECG information and the obtained blood flow change information of the first surface and the second surface; and measuring pulse wave velocity difference information based on the pulse wave velocity information to the first surface and the second surface.

The obtaining of the pulse wave velocity information to the first surface and the pulse wave velocity information to the second surface may include: obtaining pulse arrival time (PAT) information of the first surface and PAT information of the second surface based on the ECG information and the blood flow change information; and obtaining the pulse wave velocity information to the first surface and the pulse wave velocity information to the second surface based on the PAT information of the first surface and the second surface and a distance between a predetermined position of the object and the first surface and the second surface.

Each of the first optical sensor and the second optical sensor may include a light-emitting device and a light-receiving device.

The method may further include: receiving body information of the object; and estimating the distance between the predetermined position of the object and the first surface and the second surface based on the received body information.

The method may further include alternately supplying power to the first optical sensor and the second optical sensor.

The obtaining of the blood flow change information of each of the first surface and the second surface may include: measuring a first reflected light value of the first surface of the object by using the first optical sensor; measuring a second reflected light value of the second surface of the object by using the second optical sensor; and obtaining the blood flow change information of the first surface of the object and the second surface of the object based on the first reflected light value and the second reflected light value.

The method may further include: measuring a first reflected light value of the first surface of the object by using the first optical sensor; measuring a second reflected light value of the second surface of the object by using the second optical sensor; and outputting a contact correction message based on the first reflected light value and the second reflected light value.

The method may further include outputting at least one of the ECG information, the blood flow change information of the first surface of the object and the second surface of the object, and the pulse wave velocity information.

The method may further include outputting a notification message based on the pulse wave velocity difference information.

The obtaining of the pulse wave velocity information to the first surface and the pulse wave velocity information to the second surface may include: receiving an input that requests to measure a pulse wave velocity; and obtaining the pulse wave velocity information a predetermined period of time after the input is received.

The obtaining of the ECG information of the object may include: measuring a voltage of the object through the first electrode and the second electrode; and obtaining the ECG information of the object based on the measured voltage.

In an embodiment of the present invention for solving the conventional problems, a mobile device for measuring a pulse wave velocity difference may include: a first circuit unit including a first electrode that contacts a first surface of an object and a first optical sensor that contacts the first surface; a second circuit unit including a second electrode that contacts a second surface of the object and a second optical sensor that contacts the second surface; and a controller including: an electrocardiogram (ECG) information obtainer configured to obtain ECG information of the object through the first electrode and the second electrode; a blood flow change information obtainer configured to obtain blood flow change information of the first surface and the second surface by using the first optical sensor and the second optical sensor; a pulse wave velocity information obtainer configured to measure pulse wave velocity information to the first surface and pulse wave velocity information to the second surface based on the obtained ECG information and the obtained blood flow change information; and a pulse wave velocity difference measurer configured to measure pulse wave velocity difference information based on the pulse wave velocity information to the first surface and the pulse wave velocity information to the second surface.

The pulse wave velocity information obtainer may obtain pulse arrival time (PAT) information of the first surface and PAT information of the second surface based on the ECG information and the blood flow change information, and obtain the pulse wave velocity information to the first surface and the pulse wave velocity information to the second surface based on the PAT information of the first surface and the second surface and a distance between a predetermined position of the object and the first surface and the second surface.

Each of the first optical sensor and the second optical sensor may include a light-emitting device and a light-receiving device.

The mobile device may further include a user input unit configured to receive body information of the object, wherein the controller further includes a distance estimator configured to estimate the distance between the predetermined position of the object and the first surface and the second surface based on the received body information.

The controller may control power to be alternately supplied to the first optical sensor and the second optical sensor.

The controller may further include a sensor information obtainer configured to measure a first reflected light value of the first surface of the object by using the first optical sensor and a second reflected light value of the second surface of the object by using the second optical sensor, wherein the blood flow change information obtainer obtains the blood flow change information of the first surface of the object and the second surface of the object based on the first reflected light value and the second reflected light value.

The mobile device may further include an output unit, wherein the controller further includes a sensor information obtainer configured to measure a first reflected light value of the first surface of the object by using the first optical sensor and to measure a second reflected light value of the second surface of the object by using the second optical sensor, wherein the controller controls the output unit to output a contact correction message based on the first reflected light value and the second reflected light value.

The mobile device may further include an output unit, wherein the controller controls the output unit to output at least one of the ECG information, the blood flow change information of the first surface of the object and the second surface of the object, and the pulse wave velocity information.

The mobile device may further include an output unit, wherein the controller controls the output unit to output a notification message based on the pulse wave velocity difference information.

The mobile device may further include a user input unit configured to receive a request to measure a pulse wave velocity, wherein the controller controls the first circuit unit and the second circuit unit to measure the pulse wave velocity information a predetermined period of time after the request is received.

The ECG information obtainer may measure a voltage generated in the object through the first electrode and the second electrode and obtain the ECG information of the object based on the measured voltage.

### MODE OF THE INVENTION

The terms used in the present invention are selected from among common terms that are currently widely used in consideration of their function in the present invention. However, the terms may be different according to an intention of one of ordinary skill in the art, a precedent, or the advent of new technology. Also, in particular cases, the terms are discretionally selected by the applicant of the present invention, and the meaning of those terms will be described in detail in the corresponding part of the detailed description. Therefore, the terms used in the present invention are not merely designations of the terms, but the terms are defined based on the meaning of the terms and content throughout the present invention.

Throughout the specification, it will be understood that when an element is referred to as being “connected” to another element, it may be “directly connected” to the other element or “electrically connected” to the other element with intervening elements therebetween. It will be further understood that when a part “includes” or “comprises” an element, unless otherwise defined, the part may further include other elements, not excluding the other elements. Also, the terms such as “ . . . unit” and “module” used in the specification refer to units that perform at least one function or operation, and the units may be implemented as hardware or software or as a combination of hardware and software.

The term ‘mobile device’ throughout the specification may include not only a mobile device but also a case and a cover connected to or mounted on the mobile device. According to an embodiment of the present invention, examples of the mobile device may include a portable device such as, but not limited to, a mobile phone, a smart phone, a tablet, a notebook, a personal digital assistant (PDA), a portable multimedia player (PMP), a navigation system, an MP3 player, or a digital camera, and may include various other devices.

Also, according to an embodiment of the present invention, examples of the cover and the case connected to or mounted on the mobile device may include various covers and cases such as, but not limited to, a case that may cover both front and rear surfaces of a cell phone (e.g., a flip case), a case that covers only a rear surface, and a case that covers only an edge of a cell phone.

The term ‘notification message’ throughout the specification may refer to any method of notifying information about a body resistance to a user of a mobile device by displaying a text message, outputting a voice message, generating a vibration, flickering a lamp, or changing a color of a lamp.

The term ‘object’ throughout the specification may refer to a user or the body of a user.

Embodiments of the present invention will now be described more fully with reference to the accompanying drawings for one of ordinary skill in the art to be able to perform the present invention without any difficulty. The invention may, however, be embodied in many different forms and should not be construed as being limited to the embodiments set forth herein. Also, parts in the drawings unrelated to the detailed description are omitted to ensure clarity of the present invention. Like reference numerals in the drawings denote like elements.

The present invention will now be described more fully with reference to the accompanying drawings.

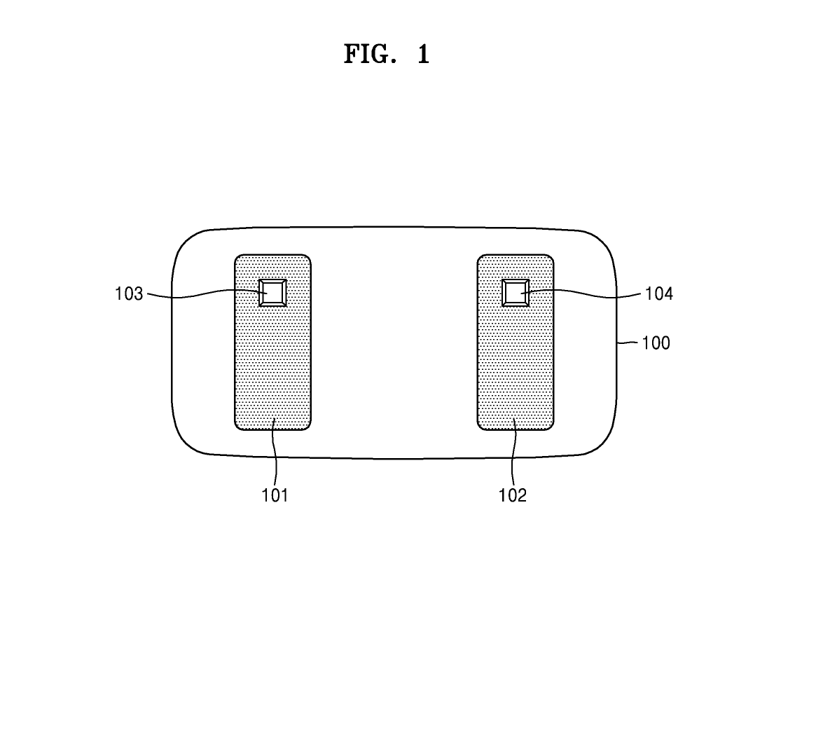

FIG. 1 is a view for explaining an arrangement of electrodes and optical sensors of a mobile device 100 according to an embodiment of the present invention.

According to an embodiment of the present invention, the mobile device 100 may be a device for measuring a pulse wave velocity. According to an embodiment of the present invention, the mobile device 100 may measure an electrocardiogram (ECG) of an object and may measure a blood flow change of the object. According to an embodiment of the present invention, the mobile device 100 may measure a pulse wave velocity of the object based on obtained ECG information and blood flow change information.

According to an embodiment of the present invention, the mobile device 10 may include two electrodes, that is, first and second electrodes 101 and 102.

According to an embodiment of the present invention, the first electrode 101 may be an electrode that contacts a first surface of the object and the second electrode 102 may be an electrode that contacts a second surface of the object. According to an embodiment of the present invention, the first surface of the object may refer to the left hand and the second surface of the object may refer to the right hand, and the object may refer to the body of a user.

In addition, according to an embodiment of the present invention, each of the first surface and the second surface may be a point of the object through which blood flows or the skin of the point. Also, the first surface and the second surface that are different from each other may each be spaced apart by a predetermined distance from the heart of the object. For example, the first surface may be the left hand of the object and the second surface may be the right hand of the object, and may each include a point that is spaced apart by a predetermined distance from the heart of the object or the skin of the point. A distance between the heart of the object and the first surface and a distance between the heart of the object and the second surface may be the same or different from each other. According to an embodiment of the present invention, the mobile device 100 may obtain ECG information by using the first electrode 101 and the second electrode 102. That is, according to an embodiment of the present invention, the mobile device 100 may measure a voltage of the object by using the first electrode 101 and the second electrode 102, and may obtain ECG information of the object based on the measured voltage and a change amount of the voltage. Also, according to an embodiment of the present invention, the first electrode 101 and the second electrode 102 may be contact electrodes or non-contact electrodes. When the first electrode 101 and the second electrode 102 are non-contact electrodes, the mobile device 100 may measure a voltage based on a capacitive coupling effect.

In addition, according to an embodiment of the present invention, the mobile device 100 may measure current through the first electrode 101 and the second electrode 102, and may obtain ECG information of the object based on the measured current and a change amount of the current.

According to an embodiment of the present invention, the mobile device 100 may include two optical sensors, that is, first and second optical sensors 103 and 104.

According to an embodiment of the present invention, the mobile device 100 may measure a blood flow change of the first surface of the object and a blood flow change of the second surface of the object by using the first optical sensor 103 that contacts the first surface of the object and the second optical sensor 104 that contacts the second surface of the object. According to an embodiment of the present invention, the first surface of the object may refer to the left hand and the second surface of the object may refer to the right hand of the object, and the object may refer to the body of the user.

According to an embodiment of the present invention, the mobile device 100 may obtain photoplethymograph (PPG) information based on the blood flow change measured by using the first optical sensor 103 and the second optical sensor 104.

According to an embodiment of the present invention, the mobile device 100 may measure a pulse wave velocity. That is, the mobile device 100 may measure a pulse wave velocity of the object based on the ECG information of the object obtained by using the first electrode 104 and the second electrode 102 and the blood flow change information obtained by using the first optical sensor 103 and the second optical sensor 104.

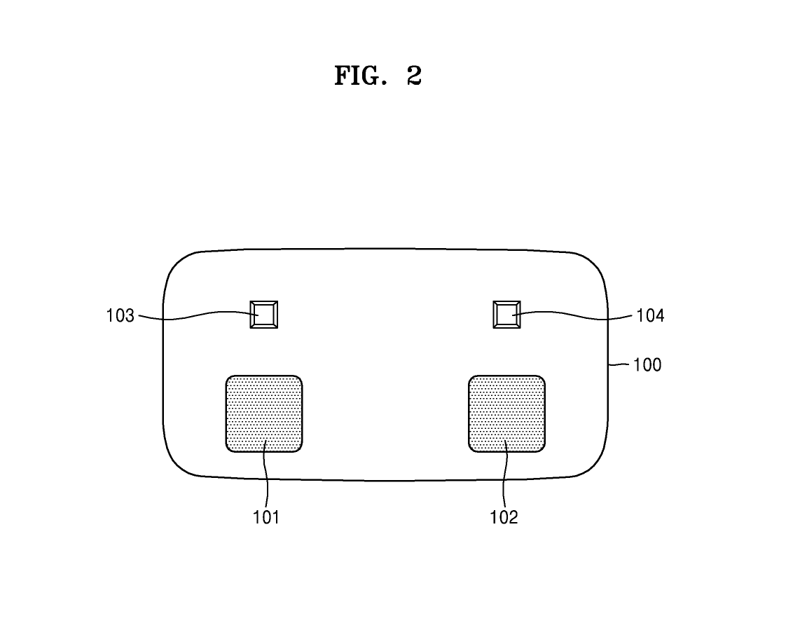

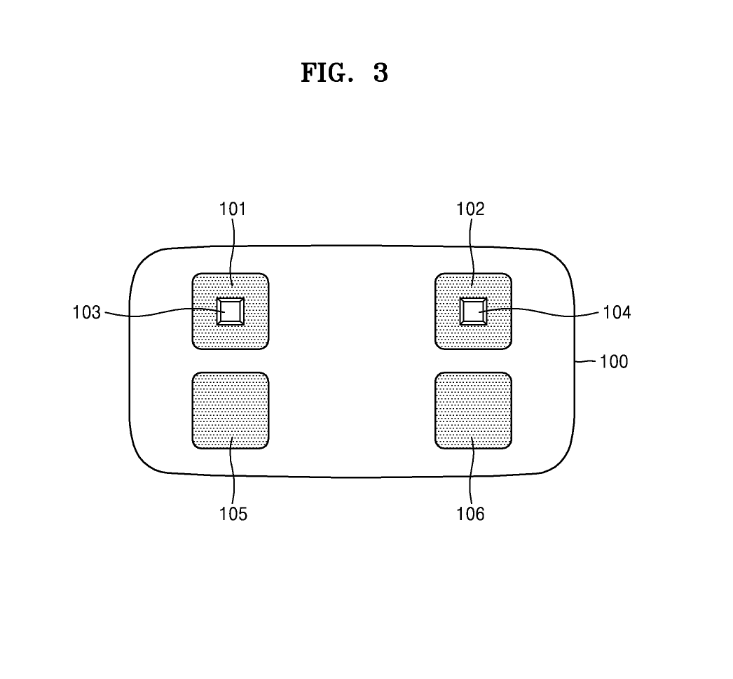

Also, as shown in FIG. 1, according to an embodiment of the present invention, the first electrode 101 may include the first optical sensor 103 and the second electrode 102 may include the second optical sensor 104. According to an embodiment of the present invention, the mobile device 100 may include four electrodes, and an arrangement of electrodes and optical sensors is not limited. Arrangements of electrodes and optical sensors included in the mobile device 100 according to additional embodiments will be explained with reference to FIGS. 2 and 3.

In addition, as described above, according to an embodiment of the present invention, the first electrode 101 and the second electrode 102 and the first optical sensor 103 and the second optical sensor 104 may be disposed on a case (or a cover) of the mobile device. Also, some or all elements needed for the mobile device 100 to measure a pulse wave velocity of the object such as the first electrode 101 and the second electrode 102 and the first optical sensor 103 and the second optical sensor 104 may be included the case of the mobile device 100.

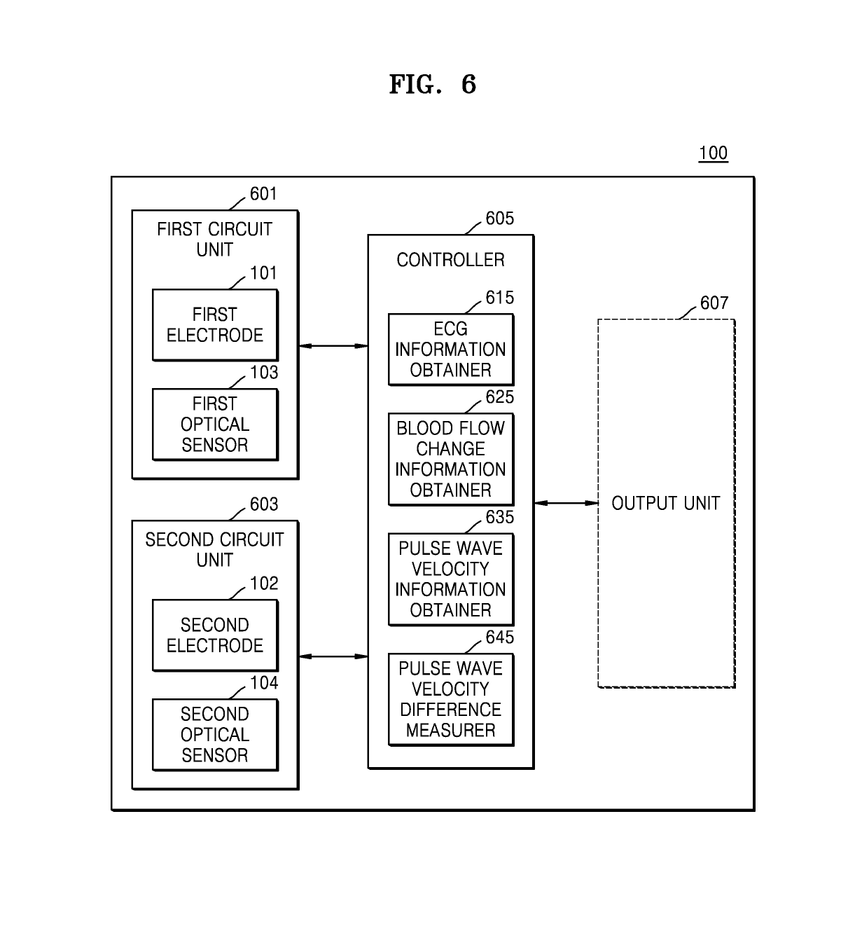

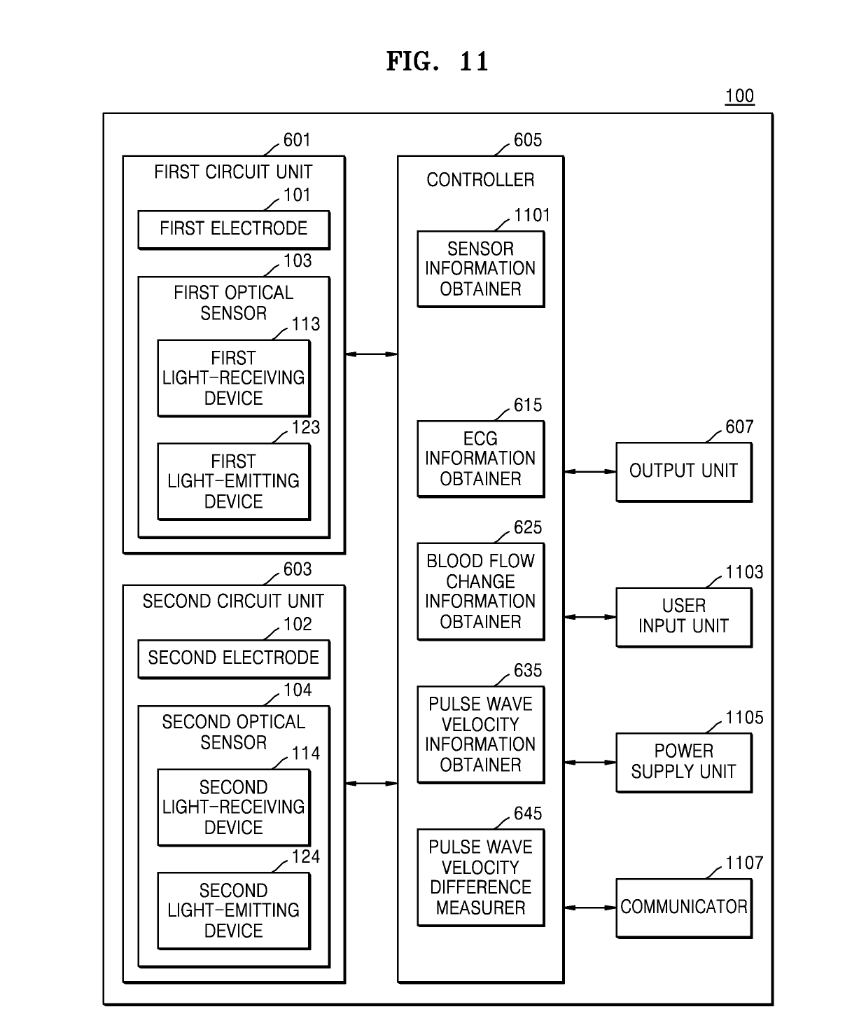

Additional elements that may be included in the mobile device 100 according to an embodiment of the present invention will be explained below in detail with reference to FIGS. 6 and 11.

FIG. 2 is a view for explaining an arrangement of electrodes and optical sensors of the mobile device 100 according to an embodiment of the present invention. That is, FIG. 2 illustrates an arrangement of electrodes and optical sensors of the mobile device 100 according to an embodiment, which is different from FIG. 1.

According to an embodiment of the present invention, the first electrode 101 and the second electrode 102 are located on a lower end portion of the mobile device 100, and the first optical sensor 103 and the second optical sensor 104 are respectively located over the first electrode 101 and the second electrode 102.

According to an embodiment of the present invention, the mobile device 100 may measure ECG information of the object and blood flow change information of the first surface and the second surface of the object by causing the index finger and the middle finger of the left hand that is the first surface of the object to contact the first electrode 101 and the first optical sensor 103 and causing the index finger and the middle finger of the right hand that is the second surface of the object to contact the second electrode 102 and the second optical sensor 104.

In addition, according to an embodiment of the present invention, the first electrode 101, the second electrode 102, the first optical sensor 103, and the second optical sensor 104 may be arranged in various shapes. The first electrode and the second electrode may be arranged to be connected to each other, or may be arranged on a flip cover of the mobile device, instead of a rear surface of the mobile device. Also, the first electrode and the second electrode may be arranged on a portion other than a portion where an antenna of the mobile device 100 is disposed in order to prevent interference with electric wave transmission/reception.

Also, according to an embodiment of the present invention, a logo placed on the mobile device 100 may be used as an electrode or an edge portion of the mobile device may be processed as an electrode so that whatever portion the user holds may be measured to improve user convenience, and optical sensors and electrodes may be arranged to be asymmetrical to each other. That is, an arrangement of electrodes and optical sensors of the mobile devices 100 is not limited.

Also, shapes of electrodes and optical sensors of the mobile device 100 are not limited. For example, although two electrodes and two optical sensors have square shapes, the electrodes and optical sensors may have circular shapes, trapezoidal shapes, triangular shapes, or may have different shapes.

In addition, the mobile device 100 may be configured so that an auxiliary display unit is disposed on a rear surface of the mobile device 100 and thus the user may read a message or information on the auxiliary display unit.

Also, according to an embodiment of the present invention, each of the first electrode 101 and the second electrode 102 of the mobile device 100 may be formed of any of various materials. For example, each of the first electrode 101 and the second electrode 102 may be formed of rubber coated with a conductive material, plastic, fiber, a ceramic material, or a metal. Also, when a metal is used, each of the first electrode 101 and the second electrode 102 may be coated with TiN, TiCN, or CrN to increase a surface conductivity and increase a scratch resistance to an electrode surface.

FIG. 3 is a view for explaining an arrangement of electrodes and optical sensors of the mobile device according to an embodiment of the present invention. That is, FIG. 3 illustrates the mobile device 100 including four electrodes and two optical sensors according to an embodiment.

As described above, the mobile device 100 according to an embodiment of the present invention may include the first electrode 101, the second electrode 102, a third electrode 105, and a fourth electrode 106. That is, the mobile device 100 may include four electrodes. Also, the mobile device may additionally include the first optical sensor 103 and the second optical sensor 104.

According to an embodiment of the present invention, the mobile device 100 may measure current generated in the object by using the first electrode 101 and the second electrode 102 and may measure a voltage applied to the objet by using the third electrode 105 and the fourth electrode 106, and vice versa.

Also, according to an embodiment of the present invention, the mobile device 100 may measure current or a voltage generated in the object by using all of the first electrode 101, the second electrode 102, the third electrode 105, and the fourth electrode 106. Functions of the first optical sensor 103 and the second optical sensor 104 included in the mobile device 100 correspond to those described with reference to FIGS. 1 and 2, and thus a detailed explanation thereof will not be given.

As described with reference to FIGS. 1 through 3, according to an embodiment of the present invention, an arrangement of electrodes and optical sensors of the mobile 100 is not limited, and the mobile device 100 may include two or more electrodes and two or more optical sensors.

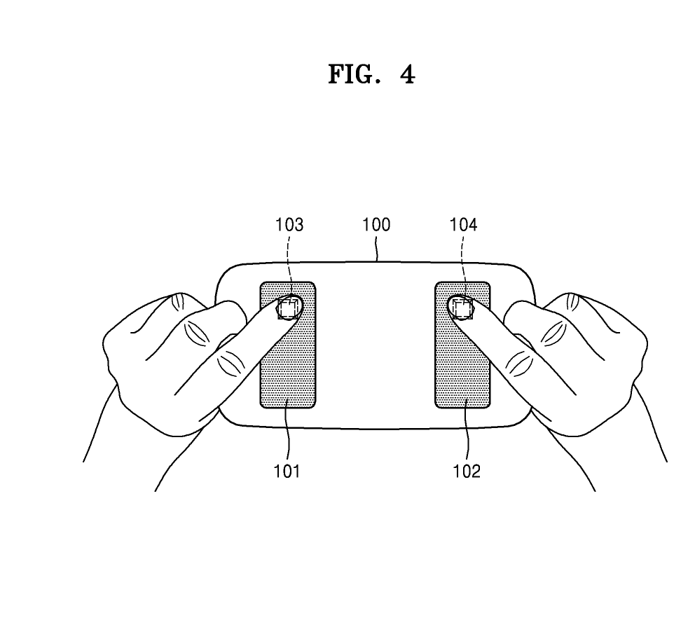

FIG. 4 is a view for explaining a posture for measuring a pulse wave velocity by using the mobile device according to an embodiment of the present invention.

Referring to FIG. 4, a posture for measuring a pulse wave velocity according to an embodiment of the present invention may include a posture made so that the first surface of the object contacts the first electrode 101 and the first optical sensor 103 of the mobile device 100 and the second surface of the object contacts the second electrode 102 and the second optical sensor 1041 of the mobile device 100. According to an embodiment of the present invention, the first surface of the object may be the point finger of the left hand of the object and the second surface of the object may be the point finger of the right hand of the object.

Although FIG. 4 illustrates a posture for measuring a pulse wave velocity, embodiments are not limited thereto. When the first optical sensor 103 and the second optical sensor 104 are not included in the first electrode 101 and the second electrode 102 as shown in FIG. 2, a posture for measuring a pulse wave velocity may be made so that the middle finger and the point finger of the left hand of the object contact the first electrode 101 and the first optical sensor 103 and the middle finger and the point finger of the right hand of the object contact the second electrode 102 and the second optical sensor 104. Even when the mobile device 100 includes four electrodes as shown in FIG. 3, a pulse wave velocity of the object may be measured by using the same posture for measuring a pulse wave velocity as that in FIG. 2.

In addition, examples of a posture for measuring a pulse wave velocity may include a posture made so that the mobile device is placed on both hands and a posture made so that electrodes and optical sensors contact at least two of body parts (e.g., the wrists or fingers) other than the hands.

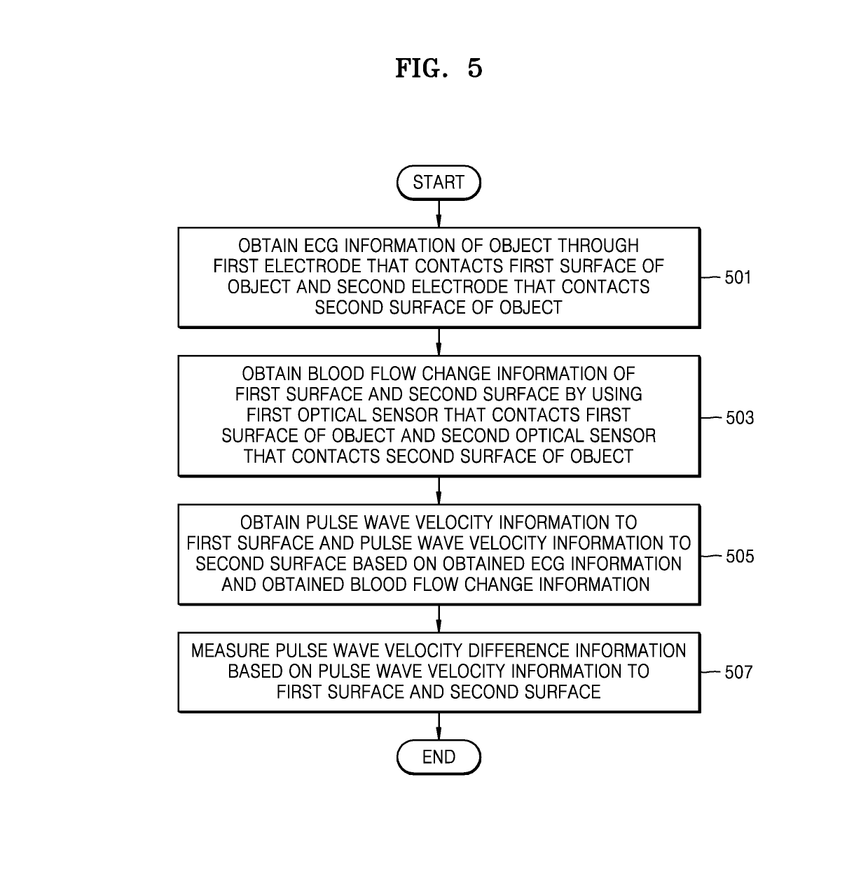

FIG. 5 is a flowchart for explaining a method of measuring a pulse wave velocity by using a mobile device according to an embodiment of the present invention.

In operation 501, a mobile device may obtain ECG information an object by using a first electrode that contacts a first surface of the object and a second electrode that contacts a second surface of the object.

According to an embodiment of the present invention, the mobile device may measure a voltage of the object by using the first electrode that contacts the first surface of the object and the second electrode that contacts the second surface of the object and may obtain ECG information of the object based on the measured voltage.

According to an embodiment of the present invention, an ECG refers to a waveform graph obtained by measuring the heart's electrical potential on a surface of the body which is obvious to one of ordinary skill in the art, and thus a detailed explanation thereof will not be given.

In operation 503, the mobile device may obtain blood flow change information of the first surface and the second surface by using a first optical sensor that contacts the first surface of the object and a second optical sensor that contacts the second surface of the object.

According to an embodiment of the present invention, blood may flow in a blood vessel of the object due to a pressure generated according to a heartbeat of the object. In particular, blood may flow up to a capillary vessel at an end of the body due to a pressure generated according to a heartbeat, and the flow of blood may have a periodicity related to the heartbeat. That is, according to an embodiment of the present invention, the mobile device may obtain information about a blood flow change according to a heartbeat by using the first optical sensor and the second optical sensor.

According to an embodiment of the present invention, an optical sensor may include a light-emitting device and a light-receiving device. That is, one set consisting of a light-emitting device and a light-receiving device may be referred to as an optical sensor.

According to an embodiment of the present invention, the mobile device may measure a first reflected light value of the first surface of the object by using the first optical sensor. That is, the mobile device may measure a first reflected light value that is a value of reflected light of the first surface of the object by emitting light to the first surface of the object by using the light-emitting device included in the first optical sensor and measuring an intensity of light that is transmitted through or reflected from the first surface of the object and is absorbed by the light-receiving device included in the first optical sensor.

Also, the mobile device may measure a second reflected light value of the second surface of the object by using the second optical sensor. The second reflected light value may be measured by using the same method as a method of measuring the first reflected light value of the first surface of the object by using the first optical sensor.

According to an embodiment of the present invention, a reflected light value may vary according to a blood flow change of the first surface or the second surface that contacts the optical sensor. That is, light absorbed by the light-receiving device included in the optical sensor of the mobile device may vary according to a blood volume or the flow of blood in the first surface or the second surface of the object that contacts the optical sensor. Accordingly, the mobile device may obtain blood flow change information of the first surface and the second surface of the object based on the first reflected light value and the second reflected light value.

According to an embodiment of the present invention, the blood flow change information may include information about a blood flow change according to time.

In addition, according to an embodiment of the present invention, the mobile device may check a contact state between the mobile device and the object by using the first optical sensor and the second optical sensor and may output a contact correction message.

As described above, according to an embodiment of the present invention, the flow of blood in the first surface and the second surface of the object may be changed according to a heartbeat of the object, and a reflected light value measured by using the optical sensor may be changed according to the changed flow of blood.

Accordingly, when a contact state between the first surface or the second surface of the object and the optical sensor or the electrode of the mobile device is poor, a measured reflected light value may have no change or may be very small. That is, a contact area between the first surface of the object or the second surface of the object and the optical sensor or the electrode of the mobile device may not be equal to or greater than a predetermined value.

According to an embodiment of the present invention, the mobile device may measure the first reflected light value of the first surface of the object by using the first optical sensor and may measure the second reflected light value of the second surface of the object by using the second optical sensor, and may output a contact correction message based on the first reflected light value and the second reflected light value.

For example, examples of the contact correction message may include a message indicating that a contact state between the first surface of the object and the second surface of the object should be checked and a message indicating that a contact state has to be corrected by laterally moving the first surface of the object and the second surface of the object. However, embodiments are not limited thereto, and examples of the contact correction message may include a message including information for correcting contact between the object and the electrode or the optical sensor.

In operation 505, the mobile device may obtain pulse wave velocity information to the first surface and velocity wave velocity information to the second surface based on the obtained ECG information and the obtained blood flow change information. According to an embodiment of the present invention, the mobile device may obtain pulse arrival time (PAT) information to the first surface of the object and PAT information to the second surface of the object based on the ECG information obtained in operation 501 and the blood flow change information of each of the first surface of the object and the second surface of the object obtained in operation 503.

As described above, since the flow of blood or the amount of blood that flows in the first surface of the object and the second surface of the object is changed due to a heartbeat, the mobile device may measure a time between a time when there is the heartbeat occurs and a time when the flow of blood or the amount of blood that flows is changed according to the obtained blood flow change information. That is, a time taken for a pulse wave to reach the first surface of the object or the second surface of the object from the heart is referred to as a PAT.

In addition, according to an embodiment of the present invention, a blood flow change at a predetermined position of the object may be measured and a PAT between the first surface of the object or the second surface of the object and the predetermined position may be measured. A PAT between a predetermined point and the first surface of the object or the second surface of the object may be referred to as a pulse transit time (PTT). When the predetermined point is the heart of the object, the PAT and the PTT may be the same.

According to an embodiment of the present invention, the mobile device may obtain pulse wave velocity information to the first surface based on a distance between a predetermined position of the object and the first surface of the object and PAT information to the first surface of the object. That is, according to an embodiment of the present invention, examples of a pulse wave velocity may include a velocity at which a pulse wave that is generated according to a heartbeat travels to a first point, and embodiments are not limited thereto.

Also, the mobile device may obtain a pulse wave velocity to the second surface of the object by using the same method as a method of obtaining the pulse wave velocity to the first surface.

Also, according to an embodiment of the present invention, the term ‘predetermined position’ of the object may be a portion of the object where the heart of the object is located. That is, a distance between the predetermined position and the first surface of the object may refer to a distance between the heart of the object and the first surface.

According to an embodiment of the present invention, the mobile device may receive body information of the object, may estimate a distance between a predetermined position of the object and the first surface based on the received body information, and may obtain a pulse wave velocity based on the estimated distance to the first surface. However, embodiments are not limited thereto, and the mobile device may estimate a distance based on stored information and may obtain a pulse wave velocity, or may receive a distance to the first surface and may obtain a pulse wave velocity based on the received distance to the first surface.

In operation 507, the mobile device may measure pulse wave velocity difference information based on the pulse wave velocity information to the first surface and the second surface. That is, according to an embodiment of the present invention, the mobile device may measure a difference in pulse wave velocity information between the first surface and the second surface based on the pulse wave velocity information to the first surface and the pulse wave velocity information to the second surface.

Also, according to an embodiment of the present invention, the mobile device may output a notification message based on the pulse wave velocity difference information. That is, the mobile device may output a notification message when a pulse wave velocity difference is equal to or greater than a predetermined value.

According to an embodiment of the present invention, the risk of developing a peripheral vascular disease, the risk of reducing a cerebral blood flow change, and the risk of dying of a heart disease or a stroke when a difference in systolic blood pressure between two arms is equal to or greater than 15 mmHg may be respectively 2.54 times, 1.5 times, and 70% higher than those when the difference in systolic blood pressure between arms is less than 15 mmHg.

That is, since a pulse wave velocity and a blood pressure have similar characteristics, since the risk of developing a cardiovascular disease in the object when a difference in pulse wave velocity between arms is equal to or greater than a predetermined value is higher than that when the difference in pulse wave velocity between arms is less than the predetermined value, the mobile device may output a notification message.

According to an embodiment of the present invention, a predetermined reference value may be determined by an input of a user, or may be determined based on information received from an external device or a server through a communicator of the mobile device.

According to an embodiment of the present invention, examples of the notification message output based on the pulse wave velocity difference information may include, but not limited to, a message warning of a disease which the object may have and a message indicating contact information of a hospital or a home page.

Also, according to an embodiment of the present invention, the mobile device may output at least one of the ECG information, the blood flow change information of the first surface of the object and the second surface of the object, and the pulse wave velocity information.

In addition, according to an embodiment of the present invention, the mobile device may receive a request to measure a pulse wave velocity and may measure a pulse wave velocity a predetermined period of time after the input is received.

Also, according to an embodiment of the present invention, the mobile device may measure a pulse wave velocity by alternately supplying power to the first optical sensor and the second optical sensor. That is, when there is only one element for controlling the optical sensor in the mobile device, the mobile device may alternately supply power to the first optical sensor and the second optical sensor in order to measure a pulse wave velocity by using the one element. The mobile device may alternately supply power to the first electrode and the second electrode by using the same method as that used to alternately supply power to the first optical sensor and the second optical sensor.

FIG. 6 is a block diagram for explaining the mobile device according to an embodiment of the present invention.

According to an embodiment of the present invention, the mobile device 100 may further include a first circuit unit 601, a second circuit unit 603, a controller 605, and an output unit 607.

According to an embodiment of the present invention, the first circuit unit 601 may include the first electrode 101 and the first optical sensor 103. Also, the second circuit unit 603 may include the second electrode 102 and the second optical sensor 104.

According to an embodiment of the present invention, the first electrode 101 may be an electrode that contacts a first surface of an object, and the first surface of the object may refer to the left hand of the object, the finger of the left hand, or the left side of the object. Also, according to an embodiment of the present invention, the first optical sensor 103 may be an optical sensor that contacts the first surface of the object.

According to an embodiment of the present invention, the second electrode 102 may be an electrode that contacts a second surface of the object, and the second surface of the object may refer to the right hand of the object, the finger of the right hand, or the right side of the object. Also, according to an embodiment of the present invention, the second optical sensor 104 may be an optical sensor that contacts the second surface of the object.

According to an embodiment of the present invention, the controller 605 may further include an ECG information obtainer 615, a blood flow change information obtainer 625, and a pulse wave velocity information obtainer 635.

According to an embodiment of the present invention, the ECG information obtainer 615 may obtain ECG information through the first electrode and the second electrode. That is, the ECG information obtainer 615 may obtain ECG information based on a voltage or current value measured through the first electrode and the second electrode.

According to an embodiment of the present invention, the blood flow change information obtainer 625 may obtain blood flow change information of each of the first surface of the object and the second surface of the object by using the first optical sensor and the second optical sensor.

According to an embodiment of the present invention, the blood flow change information obtainer 625 may measure a first reflected light value of the first surface of the object by using the first optical sensor. Also, the blood flow change information obtainer 625 may measure a second reflected light value of the second surface of the object by using the second optical sensor, and may obtain blood flow change information of the first surface and the second surface based on the first reflected light value and the second reflected light value.

According to an embodiment of the present invention, each of the first optical sensor and the second optical sensor may include a light-emitting device and a light-receiving device.

According to an embodiment of the present invention, the pulse wave velocity obtainer 635 may measure a pulse wave velocity of the object based on the obtained ECG information and the obtained blood flow change information.

According to an embodiment of the present invention, the pulse wave velocity information obtainer 635 may obtain PAT information of the first surface and PAT information of the second surface based on the ECG information and the blood flow change information.

Also, according to an embodiment of the present invention, the pulse wave velocity information obtainer 635 may obtain pulse wave velocity information to the first surface of the object and pulse wave velocity information to the second surface of the object based on the PAT information of each of the first surface and the second surface of the object and a distance between a predetermined position of the object and each of the first surface and the second surface.

According to an embodiment of the present invention, the predetermined position may be a position where the heart of the object is located, and the distance between the predetermined position and the first surface of the object and the distance between the predetermined position and the second surface of the object may be estimated based on input body information of the object as described above.

According to an embodiment of the present invention, a pulse wave velocity difference measurer 645 may measure pulse wave velocity difference information about a difference between a pulse wave velocity to the first surface of the object and a pulse wave velocity to the second surface of the object based on the pulse wave velocity information to the first surface of the object and the pulse wave velocity information to the second surface of the object.

Also, according to an embodiment of the present invention, the output unit 607 may be controlled to output a notification message based on the pulse wave velocity difference information. That is, the controller 605 may control the output unit 607 to output a notification message based on the pulse wave velocity difference information. The outputting of the notification message and the pulse wave velocity difference information are the same as those described with reference to FIG. 5, and thus a detailed explanation thereof will not be given.

According to an embodiment of the present invention, the output unit 607 may output at least one of the ECG information, the blood flow change information of the first surface of the object and the second surface of the object, and the pulse wave velocity information. That is, according to an embodiment of the present invention, the controller 605 may control the output unit 607 to output at least one of the ECG information, the blood flow change information of the first surface of the object and the second surface of the object, the pulse wave velocity information, and the pulse wave velocity difference information.

In addition, according to an embodiment of the present invention, the controller 605 may check a contact state between the mobile device 100 and the object by using the first optical sensor and the second optical sensor, and may output a contact correction message.

According to an embodiment of the present invention, the mobile device 100 may receive a request to measure a pulse wave velocity, and the controller 605 may control the first circuit unit 601 and the second circuit unit 603 to measure pulse wave velocity information a predetermined period of time after the request to measure a pulse wave velocity is received.

Also, according to an embodiment of the present invention, the controller 605 may alternately supply power to the first optical sensor 103 and the second optical sensor 104. That is, since the controller 605 controls the first optical sensor 103 and the second optical sensor 104 to be alternately driven, the controller 605 may control two or more optical sensors by using one element for driving the optical sensors, thereby efficiently measuring a pulse wave velocity by using power.

According to an embodiment of the present invention, the first circuit unit 601 and the second circuit unit 603 for measuring a pulse wave velocity may be included in a cover or a case of the mobile device, and the controller and the output unit may be included in the mobile device. That is, as described above, the term ‘mobile device’ of the present specification may include a cover or a case of the mobile device, and some elements may be included in the cover or the case of the mobile device.

Also, according to an embodiment of the present invention, the controller 605 may control a light-emitting device and a light-receiving device such as a light-emitting diode (LED) driver to be driven, may perform various operations such as an operation of converting a measured analog signal into a digital signal, an operation of converting a digital signal into an analog signal, or an operation of amplifying a signal, and may include elements for performing the various operations.

In addition, the mobile device 100 may include a power supply unit. The power supply unit may refer to a battery included in the mobile device, or an auxiliary power supply unit, instead of a battery included in the mobile device. Also, according to an embodiment of the present invention, the mobile device 100 may further include an additional element such as a communicator or a memory, which will be explained below in detail with reference to FIG. 11.

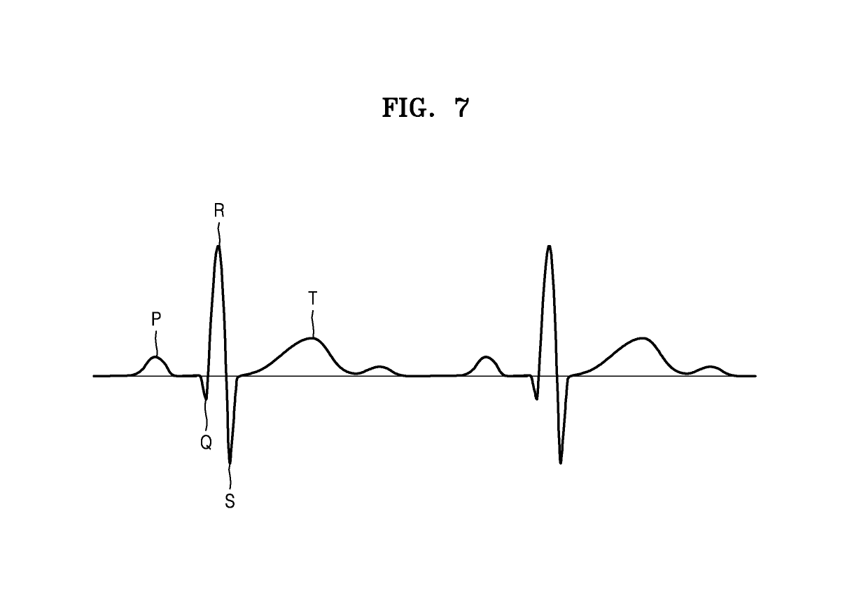

FIG. 7 is a graph for explaining ECG information according to an embodiment of the present invention.

FIG. 7 is a graph according to ECG information. That is, ECG information may refer to information about an activity of the heart of an object obtained by measuring a voltage of the object as time passes.

Various methods such as standard lead or extremity lead may be used to measure an ECG.

Referring to FIG. 7, a point P in the graph is generated when an atrium contracts and points Q, R, S, and T are generated when a ventricle contracts. The measuring of the ECG and the ECG graph are obvious to one of ordinary skill in the art, and thus a detailed explanation thereof will not be given.

According to an embodiment of the present invention, a mobile device may obtain ECG information based on a first electrode and a second electrode. The mobile device may determine a cycle of an activity of the heart of the object and a point of time when the heart contracts and a pressure is generated based on the obtained ECG information.

Also, according to an embodiment of the present invention, measured ECG information may be output as a graph similar to the ECG graph of FIG. 7.

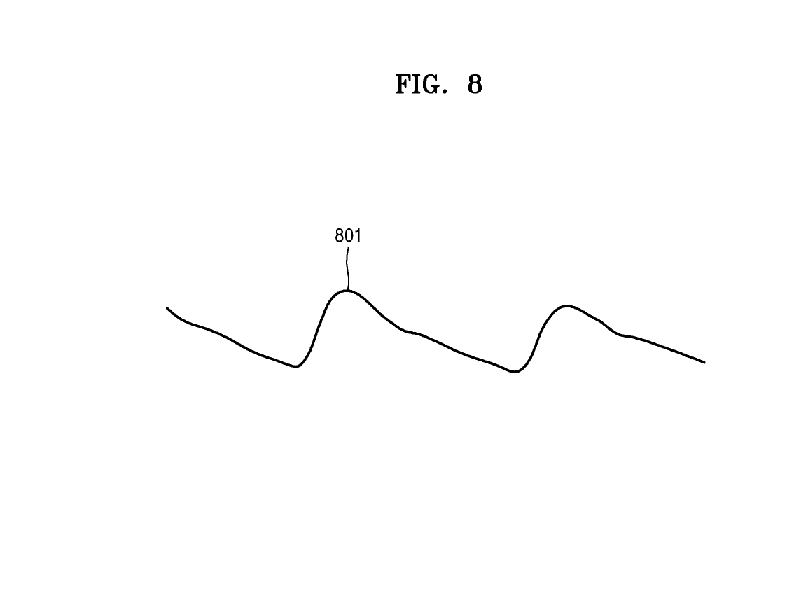

FIG. 8 is a graph for explaining pulse wave information according to an embodiment of the present invention.

According to an embodiment of the present invention, a pulse wave measured through an optical sensor may be referred to as a photoplethymograph (PPG).

A pulse wave graph of FIG. 8 may correspond to a pulse wave measured according to a blood flow change measured in a first surface or a second surface of an object. As described above, a mobile device may measure a blood flow change of the first surface and the second surface by using an optical sensor, and may measure a pulse wave in the first surface and the second surface as shown in FIG. 8 according to a result of the measurement.

According to an embodiment of the present invention, the mobile device may measure an ECG as shown in FIG. 7 by using an electrode, and may measure a pulse wave in each of the first surface and the second surface as shown in FIG. 8 by using an optical sensor. Accordingly, the mobile device may measure an interval between times when a blood flow change occurs in the first surface and the second surface of the object after a heartbeat, based on ECG information and the pulse wave.

For example, when it is assumed that a heartbeat occurs at the point R of FIG. 7, for example, a pulse wave at a point 804 of FIG. 8 may be detected according to the heartbeat at the point R. Accordingly, when it is assumed that the point R of FIG. 7 indicates an ECG state of the object 1 second after the heartbeat and the pulse wave at the point 801 of FIG. 8 is a pulse wave of the object 3 seconds after the heartbeat, the mobile device may obtain information indicating that a time taken for a pulse wave that is generated due to the heartbeat to reach the first surface is 2 seconds.

According to an embodiment of the present invention, the mobile device may measure a pulse wave velocity by using a distance to the first surface of the object and an obtained PAT.

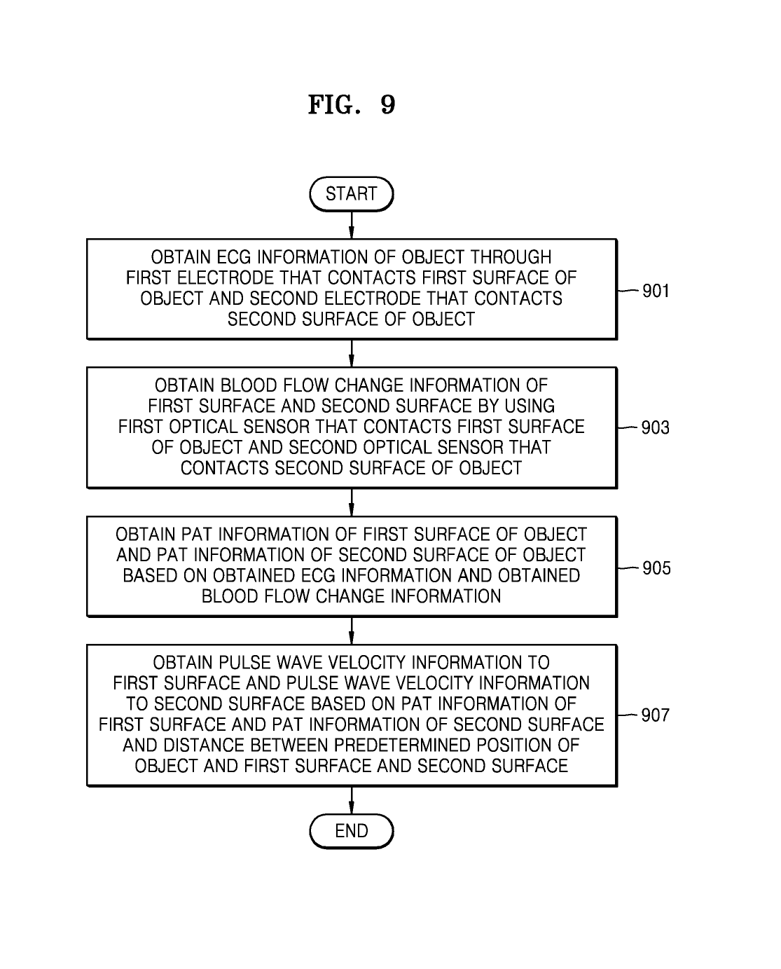

FIG. 9 is a detailed flowchart for explaining a method of measuring a pulse wave velocity according to an embodiment of the present invention.

Operations 901 through 903 correspond to operations 501 through 503 of FIG. 5, and thus a detailed explanation thereof will not be given.

In operation 905, a mobile device may obtain PAT information of a first surface and PAT information of a second surface of an object based on obtained ECG information and obtained blood flow change information.

That is, since the mobile device may obtain ECG information, blood flow change information of the first surface, and blood flow change information of the second surface as described above with reference to FIG. 8, the mobile device may obtain PAT information to the first surface of the object and PAT information to the second surface of the object based on the obtained ECG information and the obtained blood flow change information.

However, embodiments are not limited thereto. According to an embodiment of the present invention, when a predetermined position is not the heart, PTT information may be obtained.

In operation 907, the mobile device may obtain pulse wave velocity information to the first surface and pulse wave velocity information to the second surface based on the PAT information of the first surface and the PAT information of the second surface and a distance between the predetermined position of the object and the first surface and the second surface.

According to an embodiment of the present invention, the term ‘predetermined position’ of the object may refer to a portion of the object where the heart of the object is located. Also, the distance between the predetermined position and the first surface and the second surface of the present invention may be a distance between the heart of the object and the first surface and the second surface of the object. The distance between the heart and the first surface or the second surface of the object may vary according to a measurement method and a reference value.

Also, according to an embodiment of the present invention, the mobile device may determine the distance between the predetermined position of the object and the first surface and the second surface based on an input of a user, or may obtain body information of the object and may estimate the distance between the predetermined position of the object and the first surface and the second surface based on the body information. For example, the mobile device may obtain body information such as a height of the object, a shoulder width of the object, a waistline of the object, or a leg length of the object and may measure the distance between the predetermined position and the first surface and the second surface based on the body information.

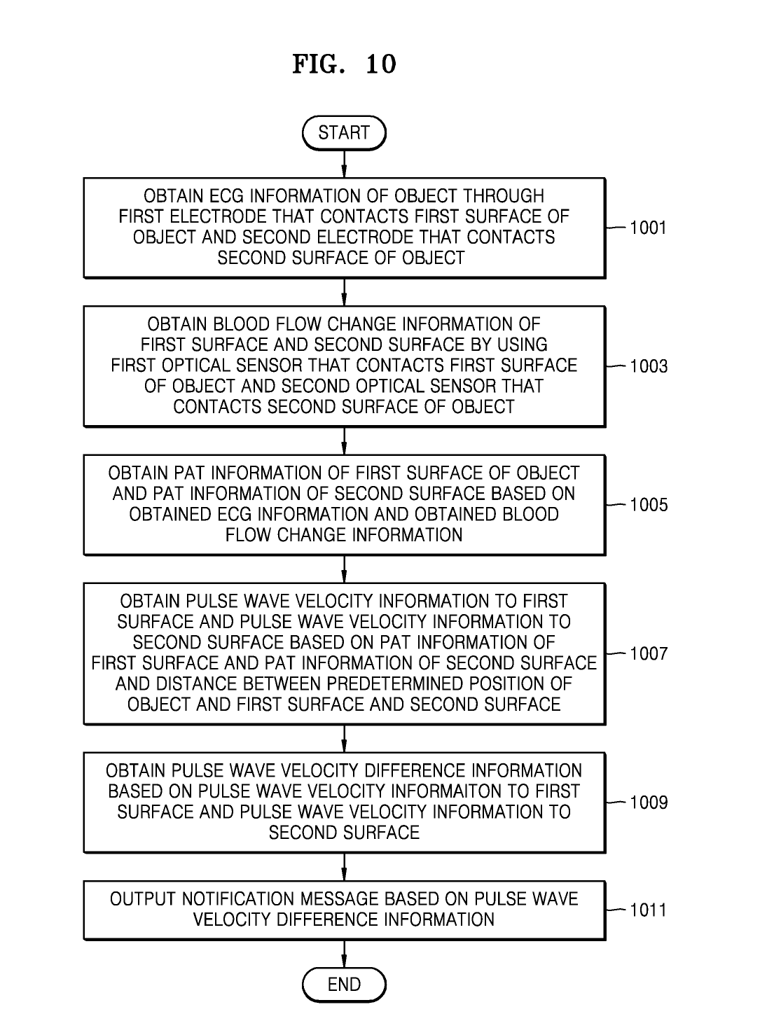

FIG. 10 is a flowchart for explaining a method of outputting a notification message according to pulse wave velocity difference information according to an embodiment of the present invention.

Operations 1001 through 1007 correspond to operations 901 through 907 of FIG. 9, and thus a detailed explanation thereof will not be given.

In operation 1009, a mobile device may obtain pulse wave velocity difference information based on pulse wave velocity information to a first surface and pulse wave velocity information to a second surface.

According to an embodiment of the present invention, since the heart of an object is located on the left side, the mobile device may obtain the pulse wave velocity difference information of the object in consideration of a position of the heart.

In operation 1011, the mobile device may output a notification message based on the pulse wave velocity difference information. For example, when the pulse wave velocity difference information is equal to or greater than a predetermined value, the mobile device may provide various information such as a warning message, information about a vascular disease, or information related to a hospital or medicine.

In addition, according to an embodiment of the present invention, the mobile device may output at least one of obtained ECG information, blood flow change information, PAT information, the pulse wave velocity information, and the pulse wave velocity difference information.

FIG. 11 is a detailed block diagram for explaining the mobile device according to an embodiment of the present invention.

According to an embodiment of the present invention, the mobile device 100 may include the first circuit unit 601, the second circuit unit 603, the controller 605, the output unit 607, a user input unit 1103, a power supply unit 1105, and a communicator 1107.

According to an embodiment of the present invention, the first circuit unit 601 may include the first electrode 101 and the first optical sensor 103. Also, the first optical sensor 103 may include a first light-receiving device 113 and a first light-emitting device 123. The first electrode 101 and the first optical sensor 103 correspond to those in FIG. 6, and thus a detailed explanation thereof will not be given.

According to an embodiment of the present invention, the second circuit unit 603 may include the second electrode 102 and the second optical sensor 104. Also, the second optical sensor 104 may include a second light-receiving device 114 and a second light-emitting device 124. The second electrode 102 and the second optical sensor 104 correspond to those in FIG. 6, and thus a detailed explanation thereof will not be given.

According to an embodiment of the present invention, the controller 605 may include a sensor information obtainer 1101, the ECG information obtainer 615, the blood flow change information obtainer 625, the pulse wave velocity information obtainer 635, and the pulse wave velocity difference measurer 645.

According to an embodiment of the present invention, the sensor information obtainer 1101 may measure a first reflected light value of a first surface of an object and a second reflected light value of a second surface of the object by using the first optical sensor 103 and the second optical sensor 104. That is, the sensor information obtainer 1101 may control the first and second optical sensors 103 and 104. For example, the sensor information obtainer 1101 may control the first and second light-emitting devices 123 and 124 to emit light having a specific intensity, may obtain reflected light values by amplifying and filtering signals received through the first and second light-receiving devices 113 and 114, and may convert an analog signal into a digital signal or may convert a digital signal into an analog signal.

For example, the sensor information obtainer 1101 may include at least of elements for controlling an optical sensor or an electrode such as an amplifier, an analog-to-digital converter, a digital-to-analog converter, and an LED driver.

In addition, the controller 605 may control the output unit 607 to output a contact correction message based on the first reflected light value of the first surface of the object and the second reflected light value of the second surface measured by the sensor information obtainer 1101.

That is, when a measured reflected light value is equal to or less than a predetermined value or a difference between information about a cycle of an ECG of the object or an input cycle and predetermined information is equal to or greater than a predetermined value, it may be determined that a contact state between the object and the electrode or the optical sensor is poor and a contact correction message for correcting the contact state may be output.

For example, when a contact area between the object and the electrode or the optical sensor is not equal to or greater than a predetermined value, a measured reflected light value may be equal to or less than a predetermined value or a difference between information about a cycle of an ECG of the object or an input cycle and predetermined information may be equal to or greater than a predetermined value.

Also, the mobile device 100 may include a pressure sensor (not shown). The sensor information obtainer 1101 may measure an intensity of a pressure applied to the electrode or the optical sensor by using the pressure sensor, and when the intensity of the pressure is equal to or greater than a predetermined value, the controller 605 may control the output unit 607 to output a contact correction message.

In addition, the mobile device may additionally include, but not limited to, an acceleration sensor, a geo-magnetic sensor, an altimeter, or a thermometer. The sensor information obtainer 1101 may control various sensors included in the mobile device 100 and may obtain information measured by using the various sensors.

According to an embodiment of the present invention, the ECG information obtainer 615, the blood flow change information obtainer 625, the pulse wave velocity information obtainer 635, and the pulse wave velocity difference measurer 645 correspond to those in FIG. 6, and thus a detailed explanation thereof will not be given. The output unit 607 also corresponds to that in FIG. 6, and thus a detailed explanation thereof will not be given.

According to an embodiment of the present invention, the user input unit 1103 may receive various pieces of information from a user. That is, the user input unit 1103 may receive body information including a height, a weight, and a gender of the object, and may receive a request to measure a pulse wave velocity. According to an embodiment of the present invention, the user input unit 1103 may further include, but not limited to, a camera, a touch pad, a touch screen, a keypad, a track ball, an electronic pen, a keyboard, and/or a mouse.

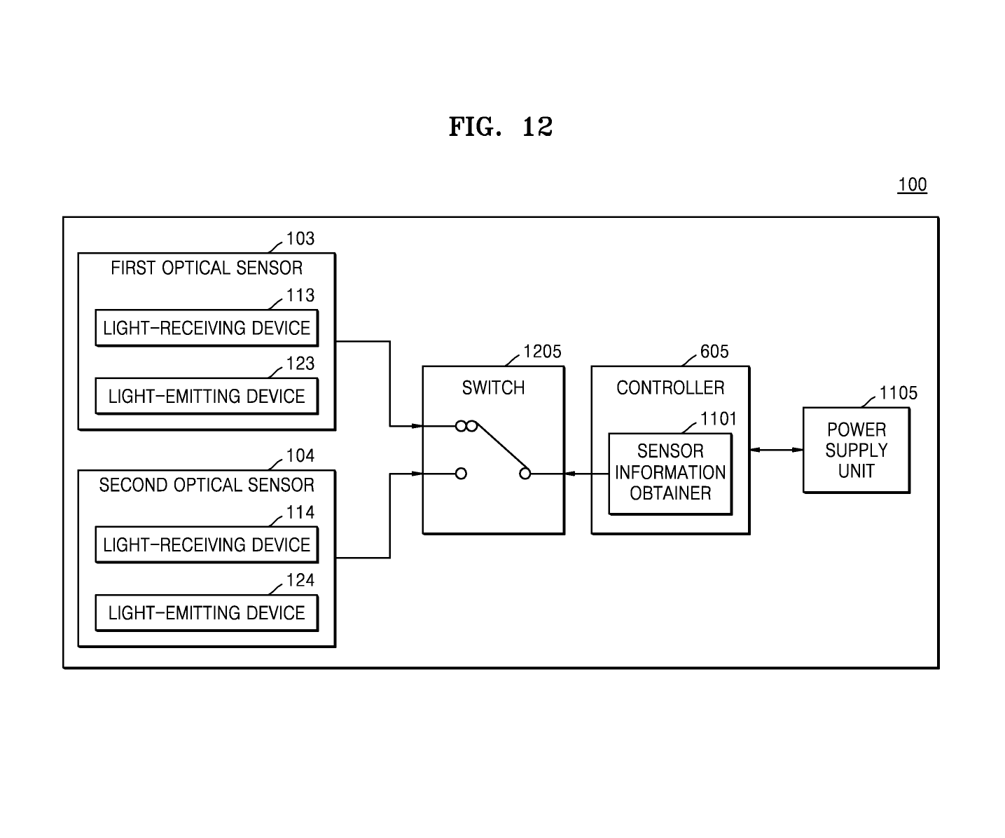

According to an embodiment of the present invention, the power supply unit 1105 may supply power to each element of the mobile device 100. Also, according to an embodiment of the present invention, the power supply unit 1105 may alternately supply power to the first optical sensor 103 and the second optical sensor 104 under the control of the controller 605, which will be explained below in detail with reference to FIG. 12.

According to an embodiment of the present invention, the communicator 1107 may perform short-range communication and long-range communication. When some elements are separately disposed in a cover or a case of the mobile device and a main body of the mobile device excluding the cover or the case, the elements may communicate with one another. For example, various pieces of information obtained by the sensor information obtainer 1101 may be transmitted to the controller.

Also, according to an embodiment of the present invention, when a calculator separate from the controller 605 of FIG. 12 exists, communication may be performed by connecting to the calculator of the mobile device through a wired interface. For example, when elements are separately disposed in the mobile device and the cover, a user interface such as an inter integrated circuit (12C), a serial peripheral interface (SPI), or a universal asynchronous receiver/transmitter (UART) between elements in the main body of the mobile device and elements in the cover of the mobile device may be established by using a Pogo pin.

In addition, according to an embodiment of the present invention, examples of wireless communication may include wireless communication according to Bluetooth, Wi-Fi, Zigbee, and Bluetooth low energy (BLE).

According to an embodiment of the present invention, the mobile device 100 may include a memory (not shown), and the memory (not shown) may store a date, a time, contact information of a user, and body information of the user.

Also, as described above, since the term ‘mobile device’ throughout the specification includes the cover and the case of the mobile device, some or all of elements of FIG. 11 may be included in the cover and the case of the mobile device. Also, since examples of the mobile device of the present specification may include a mobile device such as a cell phone, elements such as the power supply unit 1105, the communicator 1107, the memory (not shown), and the controller 605 may refer to elements included in a mobile device such as a cell phone.

That is, according to an embodiment of the present invention, the first circuit unit 601 and the second circuit unit 603 may be included in the cover of the mobile device. Also, the power supply unit 1105 and the communicator 1107 may be included in the main body of the mobile device, and the controller 605 may be a calculator in the main body of the mobile device.

For example, according to an embodiment of the present invention, when the first circuit unit 601 and the second circuit unit 603 are included in the cover of the mobile device, the first circuit unit 601 and the second circuit unit 603 may obtain ECG information and blood flow change information from the first surface of the object and the second surface of the object, and may transmit the obtained information to the controller 605 that is a calculator in the main body of the mobile device through a wired interface included in the communicator 1107 of the mobile device. Also, the controller 605 may obtain pulse wave velocity difference information based on the ECG information and the blood flow change information.

Also, according to an embodiment of the present invention, the pulse wave velocity difference information obtained by the controller 605 may be applied to the user through a display unit included in the main body of the mobile device 100, and the power supply unit 1105 may supply power to drive the first circuit unit 601 and the second circuit unit 603 provided in the cover of the mobile device.

However, embodiments are not limited thereto. According to an embodiment of the present invention, at least one of the controller 605, the power supply unit 1105, and the communicator 1107 in addition to the first circuit unit 601 and the second circuit unit 603 may be included in the cover of the mobile device.

In addition, according to an embodiment of the present invention, the cover of the mobile device may include a battery cover portion such as a cover of a battery and a flip cover portion that covers a display unit of the mobile device. In addition, according to an embodiment of the present invention, an operation of each element included in the mobile device 100 may be performed as an application is driven.

FIG. 12 is a block diagram for explaining an operation of a circuit unit of the mobile device according to an embodiment of the present invention.

FIG. 12 is a block diagram illustrating only some elements of the mobile device 100. That is, FIG. 152 illustrates only the first optical sensor 103, the second optical sensor 104, the controller 605, and the power supply unit 1105. For better understanding, a switch 1205 is additionally illustrated.

According to an embodiment of the present invention, the mobile device 100 may alternately supply power to the first optical sensor 103 and the second circuit unit 104. That is, since the sensor information obtainer 1101 in the controller 605 controls the first and second optical sensors 103 and 104 included in the mobile device 100, the sensor information obtainer 1101 may control the first optical sensor 103 and the second optical sensor 104 to be alternately driven by controlling the switch 1205.

In addition, according to an embodiment of the present invention, the mobile device 100 may alternately supply power to the first optical sensor 103 and the second optical sensor 104 without using the switch 1205. That is, the first optical sensor and the second optical sensor may be arranged so that the first optical sensor 103 operates when current flows in a first direction and the second optical sensor 104 operates when current flows in a second direction.

Since the optical sensors are alternately driven as described above, power consumption of the mobile device may be reduced. The sensor information obtainer 1101 of the mobile device 100 may control two optical sensors by using only one element for controlling the optical sensors.

The device described herein may comprise a processor, a memory for storing program data and executing it, a permanent storage unit such as a disk drive, a communications port for communicating with external devices, and user interface devices such as a touch panel, keys, and buttons. Methods using software modules or algorithms may be stored as program instructions or computer-readable codes executable on a processor in a computer-readable recording medium. Examples of the computer-readable recording medium include a magnetic storage medium (e.g., a read-only memory (ROM), a random-access memory (RAM), a floppy disk, or a hard disk), and an optical recording medium (e.g., a compact disc (CD)-ROMs, or a digital versatile disc (DVD)). The computer-readable recording medium may also be distributed over network coupled computer systems so that computer-readable code is stored and executed in a distributive manner. This medium may be read by a computer, stored in a memory, and executed by a processor.

All references, including publications, patent applications, and patents, cited herein are hereby incorporated by reference to the same extent as if each reference were individually and specifically indicated to be incorporated by reference and were set forth in its entirety herein.

For the purposes of promoting understanding of the present invention, reference has been made to the exemplary embodiments illustrated in the drawings, and specific language has been used to describe these embodiments. However, no limitation of the scope of the present invention is intended by this specific language, and the present invention should be construed to encompass all embodiments that would normally occur to one of ordinary skill in the art.

The present invention may be described in terms of functional block components and various processing steps. Such functional blocks may be realized by any number of hardware and/or software components configured to perform specified functions. For example, the present invention may employ various integrated circuit (IC) components, e.g., memory elements, processing elements, logic elements, look-up tables, and the like, which may carry out a variety of functions under the control of one or more microprocessors or other control devices. Similarly, where the elements of the present invention are implemented using software programming or software elements, the present invention may be implemented with any programming or scripting language such as C, C++, Java, or assembler language, with various algorithms being implemented with any combination of data structures, objects, processes, routines or other programming elements. Functional aspects may be implemented in algorithms that are executed on one or more processors. Furthermore, the present invention could employ any number of conventional techniques for electronics configuration, signal processing and/or control, data processing and the like. The words “mechanism”, “element”, “means”, and “configuration” are used broadly and are not limited to mechanical or physical embodiments, but may include software routines in conjunction with processors, etc.

The particular implementations shown and described herein are illustrative examples of the present invention and are not intended to otherwise limit the scope of the present invention in any way. For the sake of brevity, conventional electronics, control systems, software development, and other functional aspects of the systems may not be described in detail. Furthermore, connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the present invention unless the element is specifically described as “essential” or “critical”.

The use of the terms “a” and “an” and “the” and similar referents in the context of describing the present invention (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. Also, steps of all methods described herein may be performed in any suitable order unless otherwise indicated herein or otherwise clearly contradicted by context. The present invention is not limited to the described order of the steps. The use of any and all examples, or exemplary language (e.g., “such as”) provided herein, is intended merely to better illuminate the present invention and does not pose a limitation on the scope of the present invention unless otherwise claimed. It will be understood by one of ordinary skill in the art that numerous modifications, adaptations, and changes will be made according to design conditions and factors without departing from the spirit and scope of the appended claims.

1. A method of measuring a pulse wave velocity difference in a mobile device, the method comprising:
obtaining electrocardiogram (ECG) information of an object through a first electrode that contacts a first surface of the object and is included in the mobile device and a second electrode that contacts a second surface of the object and is included in the mobile device;
obtaining blood flow change information of the first surface and the second surface by using a first optical sensor that contacts the first surface and is included in the mobile device and a second optical sensor that contacts the second surface and is included in the mobile device;
obtaining pulse wave velocity information to the first surface and pulse wave velocity information to the second surface based on the obtained ECG information and the obtained blood flow change information of the first surface and the second surface; and
measuring pulse wave velocity difference information based on the pulse wave velocity information to the first surface and the second surface.

2. The method of claim 1, wherein the obtaining of the pulse wave velocity information to the first surface and the pulse wave velocity information to the second surface comprises:
obtaining pulse arrival time (PAT) information of the first surface and PAT information of the second surface based on the ECG information and the blood flow change information; and
obtaining the pulse wave velocity information to the first surface and the pulse wave velocity information to the second surface based on the PAT information of the first surface and the second surface and a distance between a predetermined position of the object and the first surface and the second surface.

3. (canceled)

4. The method of claim 2, further comprising:
receiving body information of the object; and
estimating the distance between the predetermined position of the object and the first surface and the second surface based on the received body information.

5. The method of claim 1, further comprising alternately supplying power to the first optical sensor and the second optical sensor.

6. The method of claim 1, wherein the obtaining of the blood flow change information of each of the first surface and the second surface comprises:
measuring a first reflected light value of the first surface of the object by using the first optical sensor;
measuring a second reflected light value of the second surface of the object by using the second optical sensor; and
obtaining the blood flow change information of the first surface of the object and the second surface of the object based on the first reflected light value and the second reflected light value.

7. The method of claim 1, further comprising:
measuring a first reflected light value of the first surface of the object by using the first optical sensor;
measuring a second reflected light value of the second surface of the object by using the second optical sensor; and
outputting a contact correction message based on the first reflected light value and the second reflected light value.

8. The method of claim 1, further comprising outputting at least one of the ECG information, the blood flow change information of the first surface of the object and the second surface of the object, and the pulse wave velocity information.

9. The method of claim 1, further comprising outputting a notification message based on the pulse wave velocity difference information.

10.-11. (canceled)

12. A mobile device for measuring a pulse wave velocity difference, the mobile device comprising:
a first circuit comprising a first electrode that contacts a first surface of an object and a first optical sensor that contacts the first surface;
a second circuit comprising a second electrode that contacts a second surface of the object and a second optical sensor that contacts the second surface; and
at least one processor configured to:
obtain ECG information of the object through the first electrode and the second electrode;
obtain blood flow change information of the first surface and the second surface by using the first optical sensor and the second optical sensor;
measure pulse wave velocity information to the first surface and pulse wave velocity information to the second surface based on the obtained ECG information and the obtained blood flow change information; and
measure pulse wave velocity difference information based on the pulse wave velocity information to the first surface and the pulse wave velocity information to the second surface.

13. The mobile device of claim 12, wherein the at least one processor is further configured to:
obtain pulse arrival time (PAT) information of the first surface and PAT information of the second surface based on the ECG information and the blood flow change information, and
obtain the pulse wave velocity information to the first surface and the pulse wave velocity information to the second surface based on the PAT information of the first surface and the second surface and a distance between a predetermined position of the object and the first surface and the second surface.

14. (canceled)

15. The mobile device of claim 13, further comprising:
a user interface configured to receive body information of the object,
wherein the at least one processor is further configured to estimate the distance between the predetermined position of the object and the first surface and the second surface based on the received body information.

16. The mobile device of claim 12, wherein the at least one processor is further configured to control power to be alternately supplied to the first optical sensor and the second optical sensor.

17. The mobile device of claim 12, wherein the at least one processor is further configured to:
measure a first reflected light value of the first surface of the object by using the first optical sensor and a second reflected light value of the second surface of the object by using the second optical sensor, and
obtain the blood flow change information of the first surface of the object and the second surface of the object based on the first reflected light value and the second reflected light value.

18. The mobile device of claim 12, further comprising:
an output interface,
wherein the at least one processor is further configured to:
measure a first reflected light value of the first surface of the object by using the first optical sensor, and
measure a second reflected light value of the second surface of the object by using the second optical sensor, and
wherein the at least one processor is further configured to control the output interface to output a contact correction message based on the first reflected light value and the second reflected light value.

19. The mobile device of claim 12, further comprising an output interface,
wherein the at least one processor is further configured to control the output interface to output at least one of the ECG information, the blood flow change information of the first surface of the object or the second surface of the object, and the pulse wave velocity information.

20. The mobile device of claim 12, further comprising:
an output interface,
wherein the at least one processor is further configured to control the output interface to output a notification message based on the pulse wave velocity difference information.

21. The mobile device of claim 12, further comprising:
a user interface configured to receive a request to measure a pulse wave velocity,
wherein the at least one processor is further configured to control the first circuit and the second circuit to measure the pulse wave velocity information a predetermined period of time after the request is received.

22. The mobile device of claim 12, wherein the at least one processor is further configured to measure a voltage generated in the object through the first electrode and the second electrode and obtain the ECG information of the object based on the measured voltage.

23. The mobile device of claim 12, wherein the first circuit and the second circuit are included in a cover portion of the mobile device.

24. A computer-readable recording medium having embodied thereon a program for executing the method of claim 1.
